(19) United States
(12) Patent Application Publication
(10) Pub. No.: US 2005/0255472 A1
(43) Pub. Date: Nov. 17, 2005

(54) Molecule analyzing method using microchannel

(76) Inventors: Kenichi Yamashita, Tosu-shi (JP); Hideaki Maeda, Tosu-shi (JP); Hajime Shimizu, Tosu-shi (JP); Masaya Miyazaki, Tosu-shi (JP); Hiroyuki Nakamura, Tosu-shi (JP); Yoshiko Yamaguchi, Tosu-shi (JP)

Correspondence Address: WENDEROTH, LIND & PONACK, L.L.P., 2033 K STREET N. W., SUITE 800, WASHINGTON, DC, 20006-1021, US

(21) Appl. No.: 10/522,137

(22) PCT Filed: Jul. 18, 2003
(86) PCT No.: PCT/JP03/09142

§ 371 Date: Jan. 19, 2005

(30) Foreign Application Priority Data
Jul. 19, 2002 (JP) 2002-211462

(51) Int. Cl. C12Q 1/68

(52) U.S. Cl. 435/6

(57) ABSTRACT

A novel method for analyzing a specimen molecule with high accuracy through a simple operation by forming a laminar flow of a specimen molecule containing solution and a complex forming molecule containing solution in a microchannel and measuring the variation of the degree of diffusion of complexes each composed of a specimen molecule and a complex forming molecule in the laminar flow. The complex forming molecule is fluorescent, and the variation of the degree of diffusion of the formed complexes is measured from the distribution of intensity of fluorescence. The degree of diffusion of the formed complexes can be quantitatively determined by comparing it with a calibration curve preliminarily defined.

### TECHNOLOGICAL FIELD

The present invention relates to a method for conducting qualitative or quantitative analysis of a specified molecule by utilizing a micro flow channel system.

### BACKGROUND TECHNOLOGY

In the studies in the field of genes, deciphering of the human genome sequences has come nearly to completion and, based on the results thereof, the center subject matters of investigations are now under shift toward the identification of the genes pertaining to gene expression, mutation, monobasic polymorphism and the like and function analysis thereof as well as toward analyses of the structures and functions of the proteins accompanying therewith.

On the other hand, by utilizing the results of these studies in series, development of technologies is now under way as directed to the services for medical aspects and welfare aspects.

As one of the means having been developed in correlation to such studies, meanwhile, there is a method for detecting living body molecules as the specimen molecules by utilizing the interaction between the molecules capable of forming a complex therewith (referred to hereinafter as the probe molecules). In this method, a qualitative detection of the presence of living body molecules or quantitative determination thereof is conducted by detecting the complex formed by a specific interaction of the probe molecules immobilized on a solid phase carrier with the living body molecules by means of the signals generated by a fluorescence substance or an electrochemically active substance labeled beforehand.

As the detecting method of the living body substances in this case, there are heretofore known, for example, a method by utilizing an electrochemical reaction [“Analytical Chemistry”, volume 72, pages 1334-1341 (2000)], a method by utilizing a quartz vibrator [“Journal of the American Chemical Society”, volume 114, pages 8299-8300 (1992)], a method by utilizing the surface plasmon resonance (published by Springer-Verlag Tokyo Inc, 1998, by Nagata and Handa, co-authors, “Real-Time Analysis of Biomolecular Interactions) and the like.

In each of the above, a nucleic acid fragment or a protein is immobilized as a probe molecule on a gold surface and brought into bonding with a living body substance or a substance related thereto having specific interaction therewith to detect and analyze the electrochemical response, frequency changes in the quartz vibrators or changes in the refractive index by the surface plasmon generated therein.

In these methods, however, a quantitative analysis with high precision requires strict control of the probe molecules to be immobilized on the solid-phase carrier body, which is unavoidably under limitation due to non-uniformity of the efficiency of immobilization of the probe molecules, poor reproducibility, complicated nature of the surface diffusion behavior of the materials on the solid-liquid interface, insufficient skill of the workers in conducting the interaction of the specimen molecules and others.

Besides, several methods are known in which immobilization onto a solid carrier is not required for the probe molecules as well as for the specimen molecules. Such methods include, for example, a method by utilizing a polymerase chain reaction (PCR) [“Proceedings of the National Academy of Sciences of the United States of America”, volume 94, page 10756 (1997)], a method by utilizing a molecular beacon [“Analytical Chemistry”, volume 72, pages 747A-753A (2000)] and the like.

The method by utilizing a PCR reaction, however, involves a reaction in which the PCR reaction per se is exponentially amplified so as to be theoretically difficult to conduct a quantitative analysis with high precision.

The method by utilizing a molecular beacon, further, is an analytical method by utilizing the fact that the probe DNA having a fluorescence site and a quenching site introduced to both terminals thereof has a nature to cause quenching of the fluorescence by bending due to the sequence of self-complementation before formation of a complex with the specimen molecules but to cause luminescence by way of the probe molecules after formation of the complex.

In order to conduct this method, however, it is unavoidable to have a defect of limited applicability only to the analyses of DNAs and other nucleic acids.

### DISCLOSURE OF THE INVENTION

The present invention has been completed under these circumstances with an object to provide, by overcoming the defects possessed by the prior art methods for the analysis in which specimen molecules or probe molecules are immobilized on a solid phase carrier, a novel method for analyzing specimen molecules, for example, a living body substance in a simple procedure still with higher accuracy.

The inventors have continued extensive investigations with regard to a method for conducting qualitative or quantitative analysis of specimen molecules by utilizing the fact that probe molecules and specimen molecules form a complex and, as a result, have arrived at a discovery that, when passed through a micro flow channel, a solution containing the probe molecules and a solution containing the specimen molecules exhibit a nature to form a laminar flow and not to form a mixture and hence specimen molecules such as living body molecules can be analyzed without immobilization onto a solid-phase carrier but still with high precision by detecting the difference in the degree of diffusion depending on the strength of the selective interaction between them, leading to completion of the present invention on the base of this discovery.

Namely, the present invention provides a method of analysis characterized by passing a solution containing specimen molecules and a solution containing molecules for complex formation through a micro flow channel in such a fashion that a laminar flow is formed, and by detecting and analyzing the changes in the degree of diffusion of the complex formed between specimen molecules and molecules for complex formation along with, in this method, a method for detecting the changes in the degree of diffusion of the complexes formed by means of the fluorescence by using molecules exhibiting fluorescence as the complex-forming molecules, and a method for quantitatively determining the concentration of the specimen molecules by comparing the degree of diffusion of the complex as formed with a calibration curve prepared in advance.

### BRIEF DESCRIPTION OF THE DRAWING

FIG. 1 is a plan view showing a model of the micro flow channel used in the inventive method.

FIG. 2 is a bar chart showing the results of Example 1.

FIG. 3 is a graph showing the relationship between the probe DNA concentration and the fluorescent intensity thereof in Example 2.

### BEST MODE FOR CARRYING OUT THE INVENTION

In the following, the present invention is described in detail.

In the method of the present invention, analysis of a specimen is conducted by simultaneously passing a solution containing probe molecules and a solution containing the specimen molecules through a micro flow channel in such a fashion as to form laminar flows and detecting the changes in the degree of diffusion of the complex formed by a specific interaction between them during flowing into the respective laminar flows depending on the extent of complex formation by means of the signals emitted from the probe molecules to analyze the results.

It is necessary that the micro flow channel used in the method of the present invention is provided on a substrate consisting of an inert material. The inert material implied here is a material, which exhibits no reactivity to the probe molecules, the specimen molecules, the solvents to be used and the complex to be formed, such as glass, quartz or silica, Si/SiO₂, magnesia, zirconia, alumina, apatite, silicon nitride and oxides of metals including titanium, aluminum, yttrium, tungsten and the like as well as ceramics including carbides, nitrides, borides, suicides and the like.

In addition, any metals, plastics and the like can be used provided that the above requirements are satisfied. With respect of the form of this base member, it can conventionally be a plate but, if so desired, those having an arch-wise form, spherical form, granular form and others can be used.

These materials can be properly selected depending on the means for selection, specimens and types of the probe molecules and the solvent but, when detection is conducted by an optical means, it is necessary to use one which exhibits sufficient transparency to the wavelength of the light used for the detecting part.

The micro flow channel of the present invention is provided on the substrate consisting of these inert materials by engraving in the size of 10 to 500 μm or, preferably 50 to 400 μm width by 10 to 500 μm or, preferably 50 to 400 μm depth.

Such a micro flow channel can be prepared by engraving, on a substrate, by a mechanical means using a machine tool such as, for example, a microdrill or, alternatively, by the photolithographic technology used in the manufacture of semiconductor integrated circuits and others to form a grave followed by adhesive bonding of another substrate thereto. The fluids flowing such an extrafine flow channel are never mixed together even if the solvents are soluble each to the other to continue flowing as forming laminar flows. Further, such an extrafine flow channel has a characteristic that the distance of diffusion of a substance is short.

At the interface between two liquids flowing through a micro flow channel, generally, assuming solubility of the solutes in the solvent, the solute causes spontaneous diffusion toward the other of the solutions but, if a specific interaction exists between the probe molecules and the specimen molecules, the diffusion of the complex therebetween can be further accelerated.

The method of the present invention provides an analytical method of a specimen by utilizing this phenomenon, by which the amount of excessive diffusion over spontaneous diffusion can be obtained enabling analysis by the amount when detection is made of the signals emitted from the functional groups introduced to the probe molecules or the specific characteristics (absorption of light having a specific wavelength, and so on) possessed by the probe molecules per se or the signals emitted by the substance selectively bonded to the complex formed or characteristics.

Accordingly, the method of the present invention can be utilized in the cases in general where the specific interaction is caused between the probe molecules and the specimen molecules. For example, a fragment of nucleic acid, when used as the probe, can be used for the detection or analysis of a fragment of nucleic acid having a specific sequence. When a protein is used as the probe, for example, a specific antibody can be detected. When various kinds of peptides having a protease inhibiting activity are used as the probe, a detection of a specific enzyme and the evaluation of the activity can be conducted. When various kinds of saccharides are used as the probe, a nucleic acid and a protein specifically recognizing the same can be detected and quantitatively determined. Various kinds of cells can be used as the probe for screening of the influences of various kinds of natural or artificial medicaments, environmental substances and the like upon organisms.

The method of the present invention can be utilized for the detection of any compounds without limitation to those, when a combination is selected in such a way that a specific interaction is generated between the probe molecules and the specimen molecules. While single molecules in a chemical meaning are employed in the above-given examples, furthermore, it is possible to have a development, besides the above, to cells and other substances in general.

With regard to the complex formation between probe molecules and specimen molecules in the method of the present invention, no skill is required for the workers as in the prior art methods. In the method of the present invention, any uncertainty in the analytical results can be excluded due to the difference in the skillfulness of the workers with respect to the complex formation between probe molecules and specimen molecules.

In the prior art methods, an experimental maneuver is required for the complex formation between probe molecules and specimen molecules such as the hybridization procedure in detecting the nucleic acid fragments, for example, so that a problem is caused relative to uncertainty of the analytical results brought about by the difference in the skillfulness of the workers in this experimental maneuver.

In contrast thereto, in the method of the present invention, skillfulness of the workers has nothing to relate since what is required is only to introduce the solutions into the flow channel with an injector and the like and the difference in the worker's skillfulness can be further excluded by utilizing a tool such as a syringe pump.

Further, in the method of the present invention, the treatment is conducted in a very small space so that it is possible to keep the concentration of the probe molecules high enough even with a small number thereof and to increase the density of the specific signals emitted from the probe molecules. According to the method of the present invention, a highly sensitive analysis can be conducted.

With respect to the length of time taken for a detection in the inventive method, which is equivalent to the time taken by the liquid introduced to the flow channel to pass therethrough, the length of time taken by a detection in the inventive method is naturally short because the time taken by the solution introduced to an ultrafine flow channel of a few hundreds μm size or so is short due to the small volume of the flow channel even if the volume of the introduced liquid is small. Though dependent on the size of the micro flow channel used and other factors, this time usually does not exceed several seconds.

In the method of the present invention, a qualitative or quantitative analysis of the specimen molecules is conducted based on the changes in the degree of diffusion of the complex formed between the specimen molecules and the probe molecules in the laminar flow. Namely, if the specimen molecules and the probe molecules have no affinity therebetween, detection can be made only for the degree of diffusion between the laminar flows based on a conventional mixing behavior in both but, when affinity is found between them, diffusion can be selectively accelerated of the complex as formed so that detection can be made as an increased degree of diffusion. Accordingly, qualitative information can be obtained for the interaction of the specimen molecules to the probe molecules by making comparison for the differences in the degrees of diffusion as mentioned.

The above-mentioned detection can be conducted usually by direct measurement of the amount of probe molecules which diffuse toward the specimen molecule solution or by measurement the amount of probe molecules at the probe molecule solution side in consideration of easy measurement. The measurement can be conducted by detecting, with a sensor, the characteristic signals emitted from the probe molecules and, it is particularly preferable to measure the intensity of the fluorescence by using, as the probe molecules, the molecules having fluorescence by introduction of a fluorescence-functional group followed by analysis of the changes in the degree of diffusion of the complex as formed.

Besides, utilizable methods include a method of using, as the probe molecules, molecules having electrochemically active functional groups introduced thereinto and determining the changes in the current, a method of measuring the changes in the absorption intensity of the probe molecules to specific ultraviolet light, visible light or infrared light and others.

In this way, analysis of the specimen molecules can be conducted without conducting chemical modification in order to obtain the specific signals to the probe molecules.

Furthermore, a quantitative determination of the concentration of the specimen molecules can be conducted by preparing beforehand a calibration curve relative to known amounts followed by making reference to the same with regard to the result of the detection.

While the above is given for the cases of measurement of the amount of the probe molecules after diffusion toward the specimen molecule solution, it is possible, if so desired, to detect the amount of the specimen molecules by determining the complex between the probe molecules and the specimen molecules after diffusion toward the probe molecule solution.

In this way, the means for detection in the method of the present invention is not particularly limited and any means are applicable, provided that the state of diffusion of the complex between the probe molecules and the specimen molecules is recognized.

In the next place, it is possible in the inventive method to introduce the solution into the micro flow channel manually with an injector connected thereto but it is advantageous to carry out under control of the liquid feed rate, liquid feed pressure and the like by a mechanical means such as a syringe pump and the like.

A detection maneuver is conducted, for example, after concurrent passing of the specimen molecule solution and the probe molecule solution through a micro flow channel and passing them over a specified distance of the flow channel. Here, at least each one kind of the specimen molecule solution and the probe molecule solution is necessary and it is possible to simultaneously obtain the data having different natures by simultaneous passing of a plurality of solutions. When the detection in this case is conducted by, for example, the fluorescent method, irradiation is made with laser beam or other lights from the excitation light source at the detecting part so as to measure the intensity of fluorescence emitted therefrom. The strong fluorescence emitted from the side of a specimen molecule solution causes the increased existing amount of the probe molecules or namely, reflects the strength of interaction between the probe molecules and the specimen molecules. In this way, information can be obtained for the presence or absence of the objective specimen or the existing amount thereof.

In the next place, the case where a detection of a DNA having the prescribed sequence is conducted by using a probe molecule introduced by a fluorescence-functional group is described in detail by way of an example.

Thus, a solution of DNA fragments as the specimen and a solution of DNA fragments having a fluorescence-functional group introduced as the probe are introduced into a micro flow channel. After flowing over a certain distance of the flow channel, irradiation is performed at the sample solution side with an excitation light such as a laser beam and the like. Absence of complementarity is concluded in the sequences between the probe DNA fragments and the specimen DNA fragments if no or only weak fluorescence is emitted therefrom. On the other hand, sequence complementarity is assumed between the probe DNA fragments and the specimen DNA fragments if the fluorescence emitted there is strong.

In this case, more accurate analysis can be accomplished with simultaneous quantitative determination of the amount of the DNA fragments having complementary sequence by making a study for the intensity of fluorescence and the degree of complementarity with known samples before undertaking the test for an unknown sample.

In this case, it is possible to conduct analysis of higher precision by using, as the probe molecules, a DNA-bondable peptide (a peptide having a structure resembling DNA with higher sequence recognizability than DNA) or an LNA (a DNA of which the 2′-position and the 5′-position of the ribose ring are connected together to exhibit high sequence recognizability) in place of the DNA. Since, different from DNAs, PNAs are soluble in organic solvents, analysis of still higher precision can be performed by using the specimen as an aqueous solution and dissolving the probe PNA in an organic solvent thereby to suppress diffusion of the specimen toward the probe solution side.

In the following, the present invention is described in more details by way of examples although the present invention is never limited by these examples.

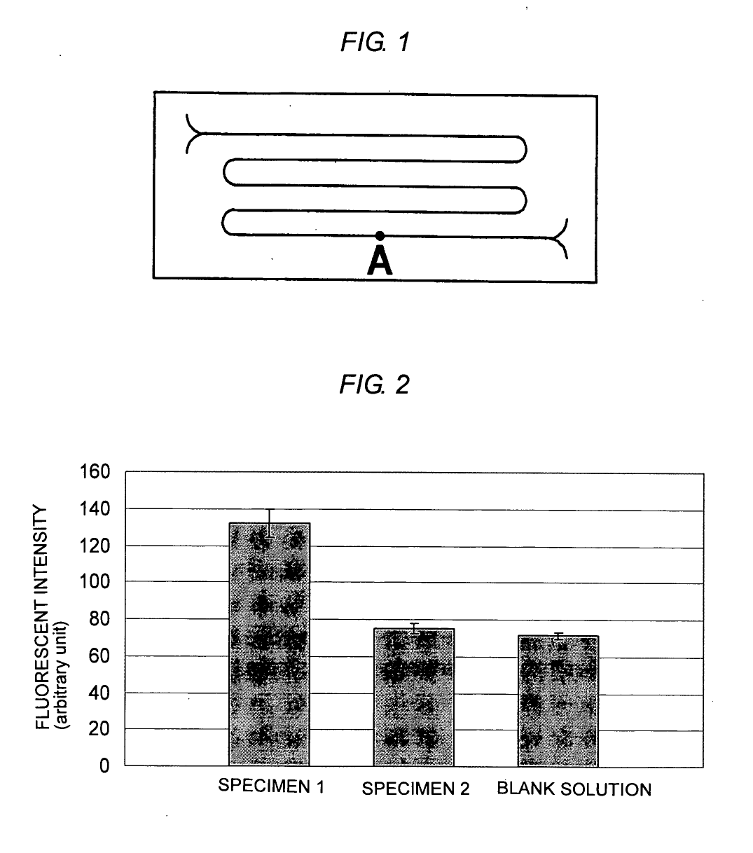

Meanwhile, the micro flow channel employed was prepared, as shown in FIG. 1, by engraving an acrylic resin plate of 70 mm by 30 mm dimensions by using a microdrill to form a channel of 360 μm width and 200 μm depth.

### EXAMPLE 1

As the probe DNA was prepared a DNA fragment having fluorecein of the fluorescent substance introduced to the 5′ terminal as expressed by the structural formula F-(5′)-AGGCTGCTCCCCGCGTGGCC-(3′) (wherein F is fluorecein).

And, as the sample DNAs were prepared two kinds of DNA fragments expressed by the structural formula (5′)-GGCCACGCGGGGAGCAGCCT-(3′) (referred to as the sample 1 hereinafter) and the structural formula (5′)-AAAAAAAAAAAAAAAAAAAA-(3′) (referred to as the sample 2 hereinafter).

Four kinds of solutions were prepared including a solution containing no DNA fragments (referred to hereinafter as the blank solution) along with the three kinds of the DNA fragment solutions. The solutions have a solution composition of 1 pmol/μl of DNA, 5 mM phosphate buffer solution (pH 7.0) and 50 mM of sodium chloride.

Three combinations including those of this probe DNA solution and the sample 1 solution, probe DNA solution and the sample 2 solution and probe DNA solution and the blank solution were each introduced to the micro flow channel at a liquid-introducing rate of 20 μl/min.

Nextly, irradiation was made with a beam of 488 nm emitted from an argon gas laser to the specimen flow channel side at the position A in FIG. 1 to cause emission of fluorescence and comparison was made for the intensities thereof. The results are shown in FIG. 2 as a bar chart. This chart shows the average values obtained by ten times measurements of the fluorescent intensities (in an arbitrary unit) and the ranges of the standard deviations are indicated with error bars. A particularly high fluorescence response was obtained only in the case of the sample 1 having a base sequence complementary with the probe DNA fragments as comparison was made with the other two. It is understood from these results that molecules having a particular interaction can be analyzed by utilizing the fact that the diffusion is accelerated by means of the interaction thereof. The results of the measurements have a variation coefficient of around 3% to indicate very high reproducibility.

### EXAMPLE 2

Mixed solutions, as the probe DNA solution, were prepared from 10 kinds of 5 mM phosphate buffer solutions (pH 7.0) containing DNA fragments having fluorescein introduced to the 5′-terminal as expressed by the structural formula F-(5′)-AGGCTGCTCCCCGCGTGGCC-(3′) (wherein F is fluorescein) in concentrations of 1 pmol/μl, 500 fmol/μl, 300 fmol/μl, 100 fmol/μl, 50 fmol/μl, 30 fmol/μl, 10 fmol/μl, 5 fmol/μl, 3 fmol/μl and 1 fmol/μl and a 50 mM aqueous sodium chloride solution.

Separately, a mixed solution was prepared from a 5 mM phosphate buffer solution (pH 7.0) containing no DNA fragments and a 50 mM aqueous sodium chloride solution.

These sample solutions were each introduced with a syringe pump to the micro flow channel of the form shown in FIG. 1 at a liquid introduction rate of 20 μl/min.

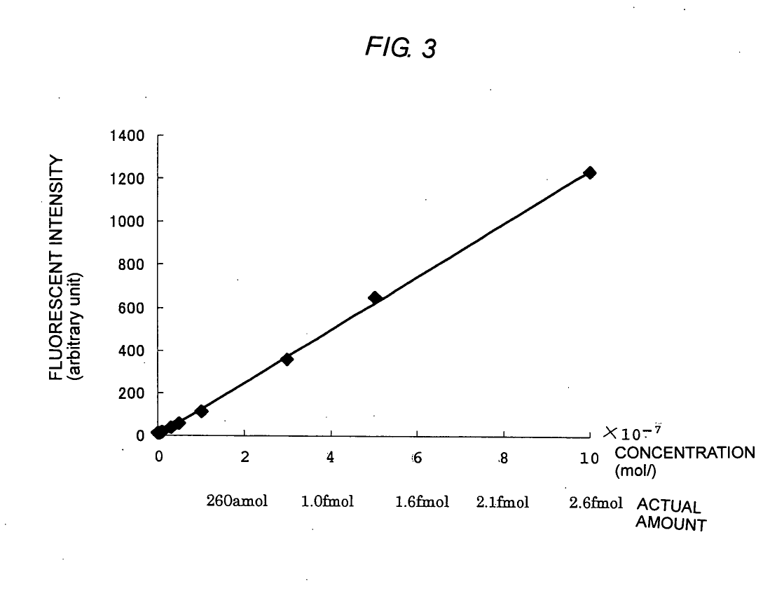

Nextly, irradiation was made with a beam of 488 nm wavelength emitted from an argon gas laser to the probe DNA flow channel side at the position A in FIG. 1 to cause emission of fluorescence and the relationship was obtained between the intensity thereof and the concentration of the probe DNA. The results are shown in FIG. 3 as a graph. FIG. 3 shows the relationship between the probe DNA concentration and the average value of the fluorescence intensities (relative value) by 10 times determinations.

In the next place, the absolute amounts of the probe DNA were calculated from the actual volume of the solution under laser beam irradiation and the concentration of the solution to be jointly given in the lower line. The thus obtained fluorescence intensity is increased by the increase in the concentration of the probe DNA or, namely the existing amount of the probe DNA and good correlation could be noted between the two variants. Though dependent on the design of the flow channel and other factors, the lower detection limit was about 10 amol in this Example.

### INDUSTRIAL UTILIZABILITY

According to the present invention, qualitative and quantitative analyses of a specimen can be conducted in high precision without immobilization on a solid phase carrier by simply introducing a probe-containing solution and a specimen-containing solution into a micro flow channel as laminar flows and measuring the degree of diffusion which is detected as a concentration of the complex between the probe molecules and the specimen molecules at a specified position. In addition, since the inventive method is performed in a simple maneuver, the error in the analytical results due to the difference in the skill of the workers can be reduced to minimum and, moreover, the molecular species of the specimens applicable is not limitative to be applicable to a wide range as an advantage.

1. A method for analyzing specimen molecules which comprises:
a step to cause flowing of a solution containing the specimen molecules and
a solution containing probe molecules capable of forming a complex with the specimen molecules in a micro flow channel in such a fashion that a laminar flow is formed;
a step to selectively promote diffusion of the complex formed according to the affinity in the laminar flow, and
a step to detect and analyze the degree of diffusion of the complex formed between the specimen molecules and the probe molecules.

2. The method of analysis described in claim 1 in which the aforementioned probe molecule is a molecule capable of emitting fluorescence.

3. The method of analysis described in claim 1 in which the detection and analysis of the degree of diffusion of the aforementioned complex are carried out by making reference to a calibration curve prepared beforehand.

4. A method for analysis of a DNA fragment which comprises:
a step to cause flowing of a solution containing a DNA fragment of a specified sequence as a specimen molecule and a solution containing a probe molecule capable of forming a complex with the specimen molecule in a micro flow channel in such a fashion that a laminar flow is formed;
a step to selectively promote diffusion of the complex formed according to affinity in the laminar flow; and
a step to detect and analyze the degree of diffusion of the complex formed between the specimen molecule and the probe molecule.
